(19) United States
(12) Patent Application Publication
(10) Pub. No.: US 2010/0057158 A1
(43) Pub. Date: Mar. 4, 2010

(54) Neurostimulation Based On Glycemic Condition

(75) Inventors: Rodolfo Rodriguez, Mira Loma, CA (US); Brian Jeffrey Wenzel, San Jose, CA (US); Fujian Qu, Sunnyvale, CA (US); Rupinder Bharmi, Canyon Country, CA (US); Karin Jarverud, Solna (SE); Taraneh Ghaffari Farazi, San Jose, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/198,791

(22) Filed: Aug. 26, 2008

(51) Int. Cl. A61N 1/365; A61N 1/36

(52) U.S. Cl. 607/22; 607/62

(57) ABSTRACT

A glycemic condition is indicated based on variance of a feature derived from cardiac electrogram data. Neurostimulation is then used to counteract a cardiac-related autonomic response to the glycemic condition. For example, stimulation of parasympathetic innervation may be used to counteract an autonomic sympathetic response that is associated with hypoglycemia or hyperglycemia. In addition, stimulation of sympathetic innervation may be used to counteract an autonomic parasympathetic response that is associated with hypoglycemia or hyperglycemia.

### TECHNICAL FIELD

This application relates generally to implantable stimulation devices and, more specifically, but not exclusively to providing neurostimulation in response to detection of hypoglycemia or hyperglycemia.

### BACKGROUND

Implantable cardiac devices may be used to treat patients with severely impaired cardiac function that results from, for example, a genetic or acquired condition. A typical implantable cardiac device may perform one or more functions relating to sensing cardiac signals and generating cardiac stimulation signals. For example, a cardiac device may sense electrical signals generated in the heart to determine whether the heart is currently in normal cardiac rhythm. If not, the cardiac device may pace the heart to maintain normal cardiac rhythm or deliver defibrillation shocks in an attempt to terminate an ongoing cardiac arrhythmia.

A significant number of patients that have severely impaired cardiac function also may have diabetes mellitus. Consequently, these patients may experience glucose excursions that result from a lack of insulin control associated with diabetes mellitus. These glucose excursions may affect the electrolytic balance in the heart which, in turn, may affect the action potentials of cardiac cells. Consequently, patients with diabetes mellitus may be more susceptible to arrhythmias and, in extreme cases, sudden cardiac death.

### SUMMARY

A summary of several sample aspects of the disclosure follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to stimulating the nervous system of a patient to improve cardiac function when the patient experiences glucose excursions. For example, stimulation signals may be applied to one or more nerves to counteract sympathetic or parasympathetic responses to changes in blood glucose levels.

The disclosure relates in some aspects to identifying blood glucose conditions in a patient by monitoring cardiac electrical activity. Here, glucose excursion may be indicated by a change in one or more features of an intracardiac electrogram (“IEGM”) or some other suitable signal. For example, hypoglycemia or hyperglycemia may be associated with a prolongation of a QT interval, an increase in QT interval dispersion, deviation in the level the ST segment, premature ventricular contractions (“PVCs”), or a change in some other cardiac feature. Accordingly, features (e.g., IEGM features) such as these may be monitored over time to identify hypoglycemia or hyperglycemia conditions.

The disclosure relates in some aspects to stimulating the nervous system if a hypoglycemia or hyperglycemia condition is indicated by a change in one or more IEGM features or other features. For example, the nervous system may be stimulated (e.g., via stimulation of parasympathetic and/or sympathetic innervation) in an attempt to mitigate any adverse cardiac events that may result from an autonomic nervous system response to hypoglycemia or hyperglycemia.

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified diagram of a stimulation device implanted in a patient;

FIG. 2 is a simplified flowchart of an embodiment of operations that may be performed to provide neurostimulation;

FIG. 3 is a simplified block diagram of an embodiment of an apparatus for providing neurostimulation;

FIG. 4 is a simplified flowchart of an embodiment of operations that may be performed to identify a glycemic condition;

FIG. 5 is a simplified diagram of an embodiment of an implantable stimulation device in electrical communication with one or more leads implanted in a patient's heart for sensing conditions in the patient, delivering therapy to the patient, or providing some combination thereof; and

FIG. 6 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

The autonomic nervous system is a complex nerve network that controls the heart rate and AV nodal conduction during varying physiologic conditions by either parasympathetic innervation or sympathetic innervation. Parasympathetic innervation typically slows the heart rate and prolongs AV conduction. In contrast, sympathetic innervation generally results in increased heart rate and shortened AV conduction. When a patient has a glycemic condition such as hypoglycemia or hyperglycemia, the patient's nervous system may provide autonomic sympathetic or parasympathetic response that may adversely affect the patient's cardiac condition. This, in turn, may increase the risk of arrhythmia and/or sudden cardiac death for the patient.

The disclosure relates in some aspects to detecting glucose excursions that may be associated with adverse cardiac events such as an increased risk of arrhythmia and/or sudden cardiac death. The disclosure also relates in some aspects to stimulating the nervous system of a patient to protect the patient's heart against glucose excursion-induced cardiac events. For example, an implantable stimulation device may comprise a closed-loop system that stimulates the autonomic nervous system to enhance, inhibit (e.g., block), or otherwise modify nervous system activity (e.g., an autonomic response). In some aspects, this stimulation may be used to counteract any adverse cardiac conditions or events that may occur in a patient with diabetes mellitus during an episode of hypoglycemia or hyperglycemia. As a result, the patient's risk of arrhythmia or sudden cardiac death during such an episode may be reduced.

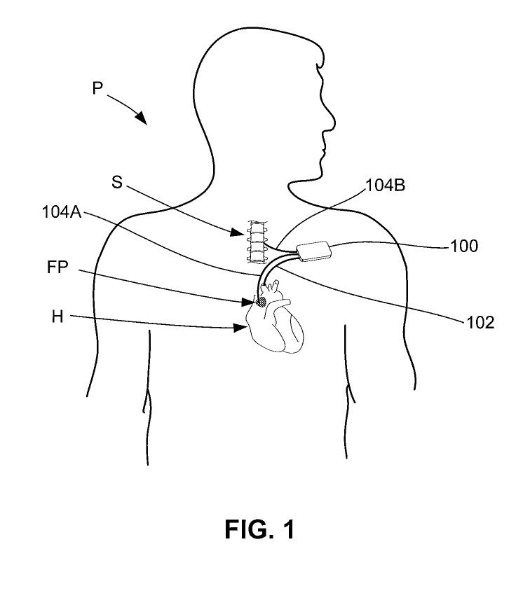

FIG. 1 illustrates a stimulation device 100 that is implanted in a patient P in a manner that enables the stimulation device 100 to monitor cardiac signals from the heart H of the patient P and apply stimulation signals to the nervous system of the patient P. To detect cardiac signals, the stimulation device 100 may be coupled to one or more implantable cardiac leads (hereafter referred to for convenience as “cardiac lead 102”). The cardiac lead 102 may, in turn, be routed from the stimulation device 100 through the patient's body and implanted within and/or on the heart H.

To supply neurostimulation signals to the nervous system, the stimulation device 100 may be coupled to one or more implantable neurological leads. A neurological lead may be routed from the stimulation device 100 through the patient's body and implanted at one or more cites within patient that provide access to the nervous system. For example, FIG. 1 illustrates that a neurological lead 104A may be implanted adjacent to (e.g., on or near) one or more nerves at a fat pad FP on an epicardial surface of the heart H. FIG. 1 also illustrates that a neurological lead 104B may be implanted adjacent to one or more nerves at the spine S of the patient P. For convenience, one or more neurological leads may be referred to in the discussion that follows simply as “the neurological lead 104.”

As will be discussed in more detail below, the location at which neurostimulation is provided may depend on the type of innervation being applied. For example, parasympathetic innervation involves brainstem nerves and the cranial nerves projecting to the vagus nerve and to the cardiac ganglia. Conversely, sympathetic efferent innervation of the heart involves central ganglia with preganglionic processing in T1-T5 positions of the spinal cord and then projections to the stellate ganglia and cardiac ganglia. Cardiac ganglia reside on epicardial fat pads near the right pulmonary vein-left atrium, inferior vena cava-left atrium, and superior vena cava.

Accordingly, to provide a parasympathetic response that counteracts an autonomic sympathetic response to a glycemic condition, the neurological lead 104 may be implanted at (e.g., on or near) the T1-T5 positions of the spinal cord or at epicardial fat pads. In addition, to stimulate a sympathetic response that counteracts an autonomic parasympathetic response to a glycemic condition, the neurological lead 104 may be implanted on or near epicardial fat pads or the vagus nerve. The neurological lead 104 also may be implanted in a manner that enables stimulation of other cardiac nerves (e.g., the great cardiac nerve) to provide stimulation of parasympathetic or sympathetic innervation. Also, multiple neurological leads 104 may be employed in some embodiments to provide stimulation of parasympathetic innervation and/or sympathetic innervation.

A stimulation signal may be applied to a nerve in various ways. For example, in some embodiments a neurological lead (e.g., one or more electrodes on a proximal end of the lead) may be directly attached to a nerve such that stimulation signals are directly coupled to the nerve. In some embodiments a neurological lead (e.g., one or more electrodes of the lead) may be placed near a nerve such that stimulation signals are indirectly coupled (e.g., radiated) to the nerve via surrounding matter (e.g., tissue, fat pad, spinal cord).

Referring again to FIG. 1, the stimulation device 100 may be implanted a various locations within the patient P. For example, in some embodiments the stimulation device 100 may be implanted subcutaneously in the pectoral region of a patient's chest as shown in FIG. 1.

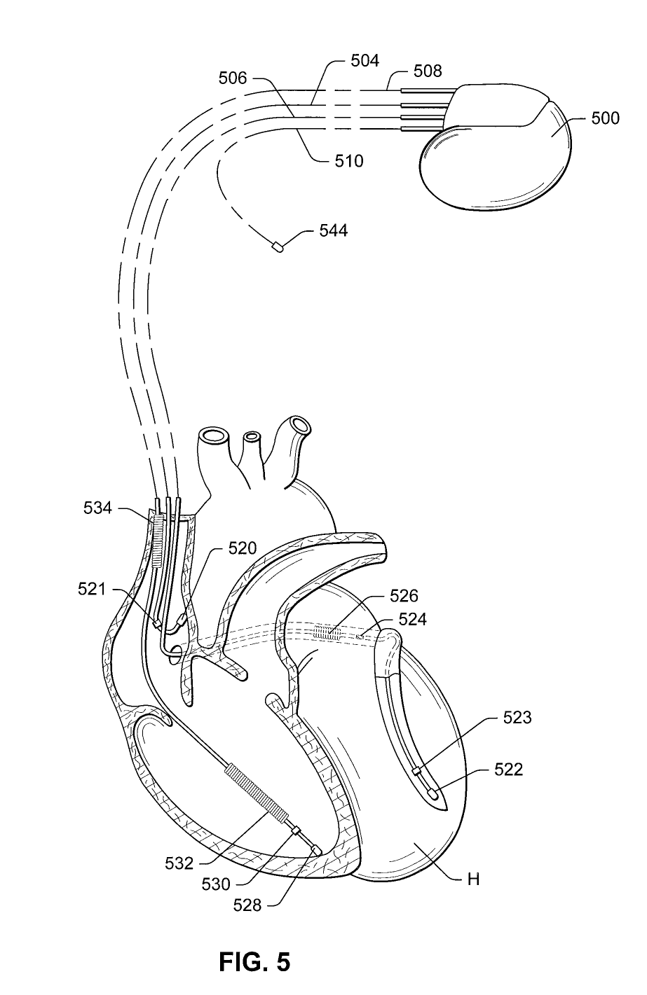

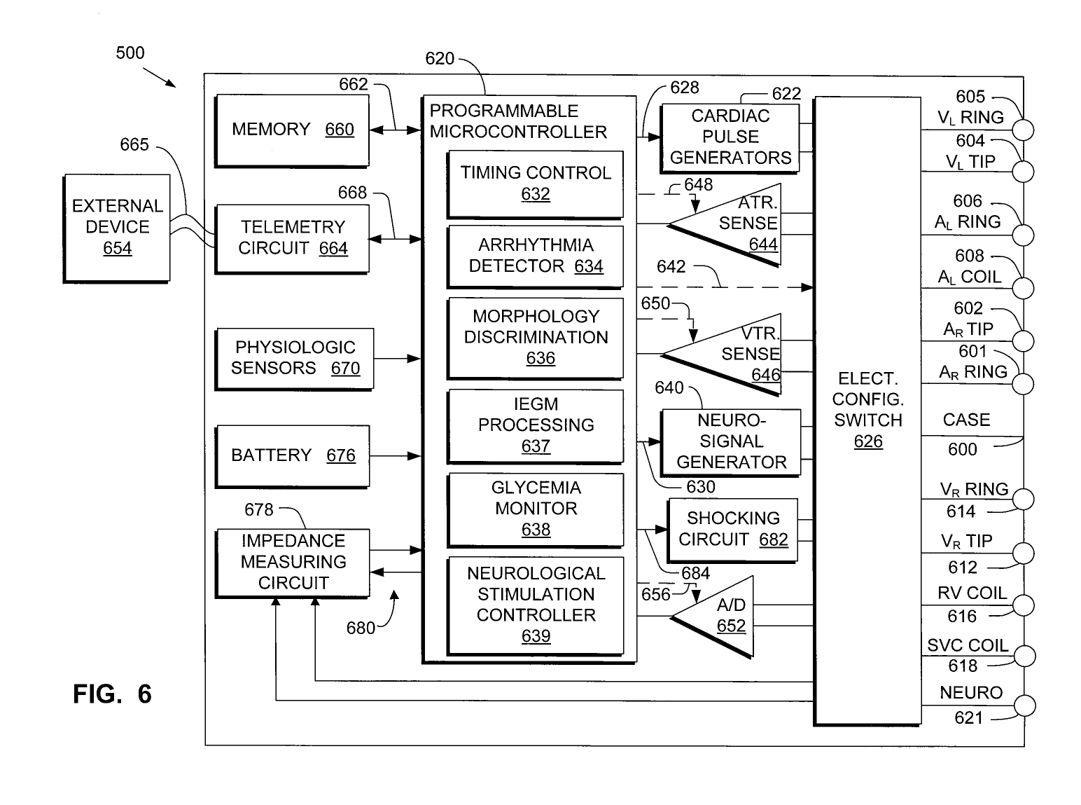

The stimulation device 100 may take various forms. For example, in some embodiments the stimulation device 100 may comprise a dedicated neurostimulation device. In some embodiments the stimulation device 100 may comprise an implantable cardioverter defibrillator (“ICD”) or some other type of cardiac device that also includes neurostimulation components and is configured to connect to the neurological lead 104. In this case, the cardiac leads 102 may be used for monitoring cardiac activity and applying stimulation signals (e.g., pacing pulses and/or shocks) to the heart H as will be described in more detail below in conjunction with FIGS. 5 and 6.

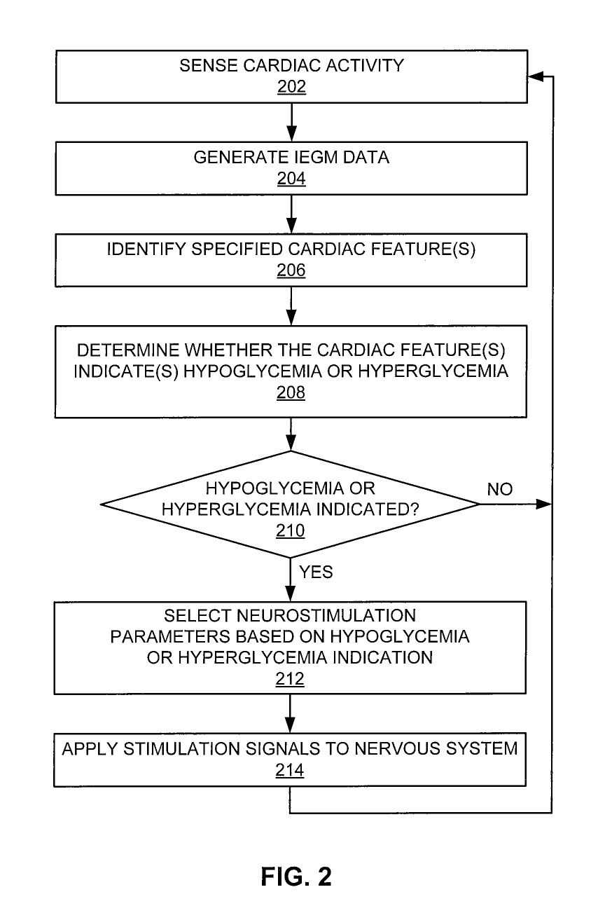

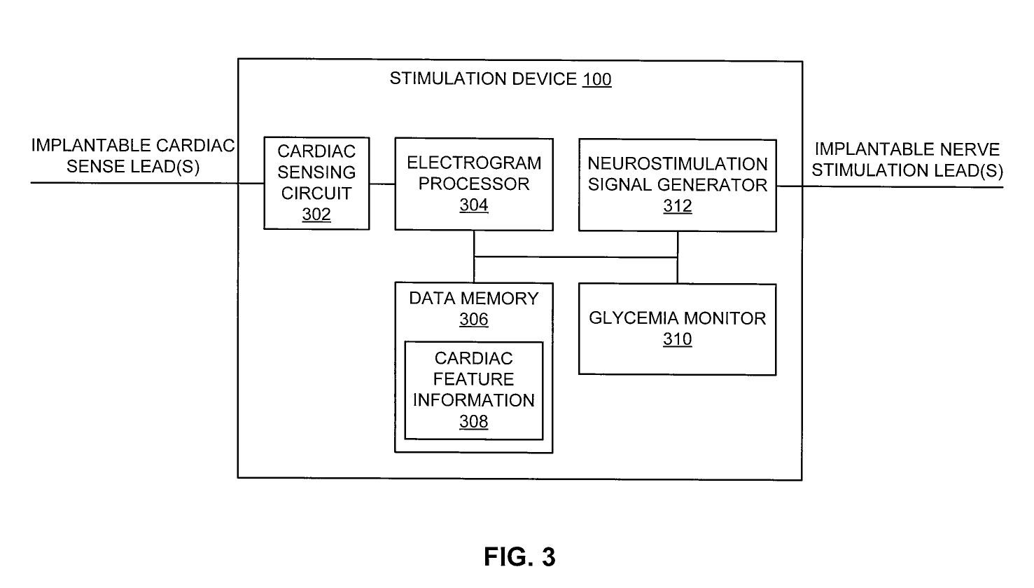

Referring to the flowchart of FIG. 2, several sample operations relating to neurostimulation that may be performed by the stimulation device 100 will be described. For convenience, the operations of FIG. 2 (or any other operations discussed or taught herein) may be described as being performed by specific components (e.g., the components depicted in FIG. 3). It should be appreciated, however, that these operations may be performed by other types of components and may be performed using a different number of components. It also should be appreciated that one or more of the operations described herein may not be employed in a given implementation.

As represented by block 202 of FIG. 2, the stimulation device 100 may be configured to sense cardiac activity. For example, as shown in FIG. 3, the stimulation device 100 may include a cardiac sensing circuit 302 that senses cardiac electrical signals. The cardiac sensing circuit 302 may include or operate in conjunction with one or more implantable cardiac leads (e.g., lead 102) and include appropriate amplification and filtering components to generate cardiac signals corresponding to the cardiac activity.

As represented by block 204 of FIG. 2, the stimulation device 100 may generate electrogram data (e.g., IEGM data) based on the sensed cardiac activity. For example, an electrogram processor 304 (FIG. 3) may process the cardiac signals provided by the sensing circuit 302 to provide IEGM data representative of cardiac events such as P-waves, QRS complexes, T-waves, and so on for a series of cardiac cycles. The electrogram processor 304 may then store this IEGM data in a data memory 306 for subsequent processing. In implementations where the stimulation device 100 is an implantable cardiac stimulation and/or monitoring device, IEGM data may be generated on a continual basis for use in cardiac pacing or other operations. Consequently, in these implementations the acquisition of IEGM data for neurostimulation operations may simply involve retrieving previously collected IEGM data from the data memory 306.

As represented by block 206, the electrogram processor 304 may identify (e.g., extract) one or more cardiac features from the IEGM data that may be used (as discussed below) to determine a current glycemic condition of a patient. These cardiac features may relate to, for example, timing, amplitude, signal levels, and area under the curve for one or more cardiac events. In addition, the electrogram processor 304 may collect this cardiac feature information over one or more cardiac cycles. As shown in FIG. 3, the electrogram processor 304 may store this cardiac feature information 308 in the data memory 306 for subsequent processing.

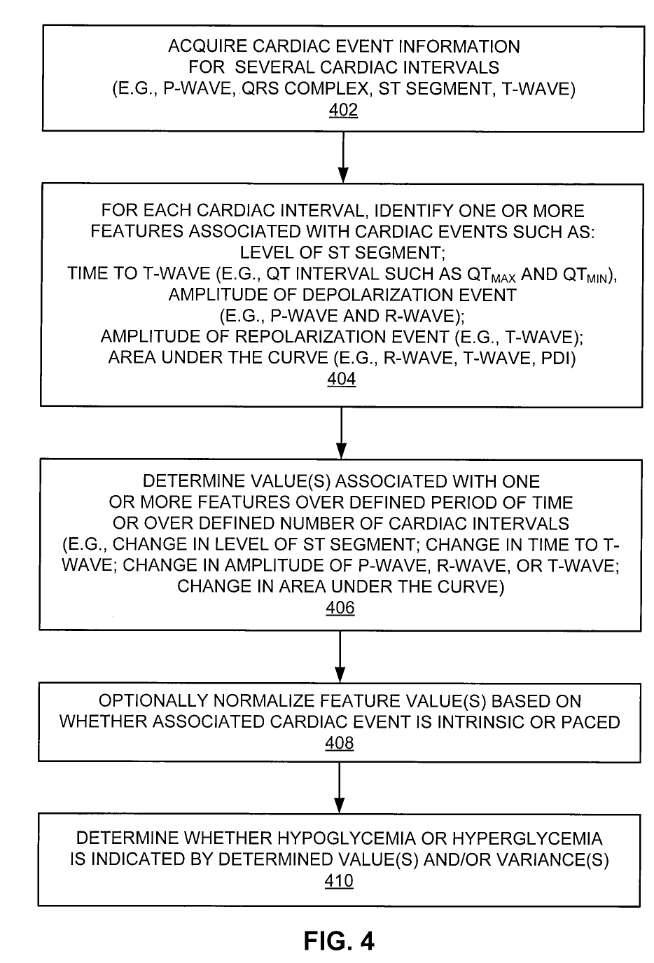

As represented by block 208, the stimulation device 100 may include a glycemia monitor 310 that processes the IEGM data (e.g., the cardiac feature information 308) to determine whether a hypoglycemia or hyperglycemia condition is indicated. Such a process may take various forms and may be based on various cardiac features. Several examples of such features and associated glycemia determination operations will be described in conjunction with FIG. 4.

As represented by block 402, in some aspects hypoglycemia or hyperglycemia may be indicated based on analysis of cardiac events such as P-waves, QRS complexes (e.g., R-waves), ST segments, and T-waves. Accordingly, the processing of IEGM data described above at block 206 may initially involve acquiring cardiac event information for one or more of the above event types over several cardiac cycles (e.g., by collecting event information for several consecutive heartbeats). It should be appreciated that hypoglycemia or hyperglycemia may be detected using other techniques (e.g., external sensor-based detection) that acquire other information (e.g., electrocardiogram data).

As represented by block 404, in some aspects hypoglycemia or hyperglycemia may be indicated based on particular features of these cardiac events. Accordingly, the processing of IEGM data at block 206 also may involve identifying one or more of these features from the acquired cardiac event information. For example, in some aspects a glycemic condition may be indicated by a change in the level of the ST segment. In some aspects a glycemic condition may be indicated by a change in the time to a T-wave. This T-wave time may comprise, for example, a QT interval relating to the time duration from a particular point in the QRS complex (e.g., the beginning of ventricular depolarization) to a particular point in the T-wave. Examples of such intervals include the time to the minimum T-wave amplitude (T_{MIN}), the time to the maximum T-wave amplitude (T_{MAX}), and the time to the T-wave centroid. In some aspects a glycemic condition may be indicated by an amplitude of an atrial depolarization event (e.g., a P-wave), a ventricular depolarization event (e.g., an R-wave), or a ventricular repolarization event (e.g., a T-wave). In some aspects a glycemic condition may be indicated by the “area under the curve” relating to a cardiac event (e.g., area under an R-wave, area under a T-wave, or a paced depolarization integral relating to the area under an evoked response curve).

As represented by block 406, in some aspects hypoglycemia or hyperglycemia may be indicated based on the value of a particular feature at a given point in time or based on a variance associated with the value of a feature (e.g., the magnitude of the change in the value or the dispersion of the value) over time. In some cases a particular value (e.g., an absolute value) associated with a feature may be calculated based on several samples of that value. For example, a given value may be calculated as the average (e.g., mean) value of the feature over a defined period of time or a defined number of cardiac cycles. Similarly, a variance in a given value may be calculated based on a change in an average (e.g., mean) value over time or based on the average dispersion over time.

Several examples of values that the electrogram processor 304 may determine to identify a glycemic condition are described at block 406. For example, in some aspects a glycemic condition may be indicated by a change in the level of the ST segment, a change in a time to the T-wave, a change in an amplitude of a P-wave, an R-wave, or a T-wave, or a change in an area under the curve.

As represented by block 408, in some implementations the electrogram processor 304 may normalize the acquired feature information to account for differences between features derived from an intrinsic cardiac event and features derived from a paced cardiac event. Here, the morphology of some types of cardiac events may be different depending on whether the event is intrinsic or was evoked by pacing. Consequently, under similar glycemic conditions, a given feature value (e.g., a QT interval, an ST segment level, etc.) may be different (e.g., larger or smaller in magnitude) when the cardiac event is due to pacing versus when the event is intrinsic. By normalizing these feature values, all of the information collected over a period of time may efficiently be used to identify a glycemic condition, even if the information is based on a combination of intrinsic and paced events.

In some embodiments, a determination may be made as to whether a given event is intrinsic or paced (e.g., based on whether the device 100 transmitted a cardiac pacing pulse) as the cardiac event information is acquired. Based on this determination, a feature value generated at block 406 may be normalized so that a similar value is provided for similar glycemic conditions regardless of whether the underlying cardiac event was intrinsic or paced. Here, the appropriate normalization factor for a given feature may be determined based on empirical tests or determined in some other manner.

As represented by block 410, the glycemia monitor 310 determines whether the patient has a normal glucose level or is experiencing either hypoglycemia or hyperglycemia based on one or more of feature values. For illustration purposes, several examples of feature changes that indicate hypoglycemia or hyperglycemia will be briefly described.

In some aspects a glycemic condition may be indicated by a variance associated a T-wave-based interval. For example, hypoglycemia may be indicated when there is an increase in the duration of the QT interval (e.g., an increase in the mean QT interval) over a defined period of time (e.g., two minutes). Here, a T-wave-based interval may relate to, for example, QT_{MAX}, QT_{MIN}, QT_{CENTROID}, or some other time to T-wave interval.

In some aspects hypoglycemia may be indicated by an increase in the dispersion of QT intervals over a defined period of time (e.g., two minutes). Thus, in this case, the glycemia monitor 310 may track the value (e.g., the mean value) of the QT interval over time to obtain a set of dispersion values.

In some aspects hypoglycemia may be indicated by a variance associated with R-wave amplitude. For example, the variance of the R-wave amplitude over a defined period of time (e.g., two minutes) may increase when the blood glucose level decreases.

In some aspects hypoglycemia may be indicated by a relatively significant deviation in the level of the ST segment along with an increase in QT_{MAX} and QT_{MIN}. Thus, in this case, the glycemia monitor 310 may analyze multiple features to make a determination as to whether a hypoglycemia condition exists.

In some aspects hyperglycemia may be indicated by a relatively significant deviation in the level of the ST segment when there is relatively little or no change in QT_{MAX} and QT_{MIN}. Thus, in view of this case and the case of the preceding paragraph, the glycemia monitor 310 may distinguish a hypoglycemia condition from a hyperglycemia condition based on the values of these features.

In some aspects hyperglycemia may be indicated by a relatively significant increase in the absolute value of the amplitudes of atrial depolarization events, by a relatively significant rate of change in the amplitudes of atrial depolarization events over time, or by a relatively significant beat-to-beat change in the amplitudes of atrial depolarization events. In contrast, there may be no significant change in these parameters for a hypoglycemia condition. Thus, in some aspects, the glycemia monitor 310 may distinguish a hypoglycemia condition from a hyperglycemia condition based on these amplitude features.

In some aspects hyperglycemia may be indicated by a relatively significant increase in the absolute value of the amplitudes of ventricular depolarization events, by a relatively significant rate of change in the amplitudes of ventricular depolarization events over time, or by a relatively significant beat-to-beat change in the amplitudes of ventricular depolarization events. In contrast, hypoglycemia may be indicated by a more moderate increase in the absolute value of the amplitudes of ventricular depolarization events, by a less rapid rate of change in the amplitudes of ventricular depolarization events over time, or by a less rapid beat-to-beat change in the amplitudes of ventricular depolarization events. Thus, the glycemia monitor 310 also may distinguish a hypoglycemia condition from a hyperglycemia condition based on these amplitude features.

In some aspects hypoglycemia may be indicated by a relatively significant increase in the absolute value of the amplitudes of ventricular repolarization events, by a relatively significant rate of change in the amplitudes of ventricular repolarization events over time, or by a relatively significant beat-to-beat change in the amplitudes of ventricular repolarization events. In contrast, there may be no significant change in these parameters for a hyperglycemia condition. Thus, the glycemia monitor 310 may distinguish a hypoglycemia condition from a hyperglycemia condition based on these amplitude features.

In some embodiments, the glycemia monitor 310 may distinguish a hypoglycemia condition from a hyperglycemia condition based on amplitude characteristics associated with the several of the above depolarization and repolarization events. For example, hyperglycemia may be indicated based on a significant and rapid increase in P-wave amplitude, a significant and rapid increase in QRS complex amplitude, and a lack of significant increase in T-wave amplitude. Conversely, hypoglycemia may be indicated based on a significant and rapid increase in T-wave amplitude, a moderately rapid increase in QRS complex amplitude to moderately elevated levels, and a lack of significant increase in P-wave amplitude.

In some embodiments, the determination of whether a hypoglycemia condition or a hyperglycemia condition exists may involve analysis of other cardiac features. For example, in some aspects hypoglycemia may be indicated by an increase in the paced depolarization interval (“PDI”) over a relatively short period of time. In some aspects a glycemic condition may be indicated by a change in the area under an R-wave and/or a T-wave. In some aspects a glycemic condition may be indicated by changes in heart rate or changes associated with premature ventricular contractions.

Referring to block 210 of FIG. 2, if neither hypoglycemia nor hyperglycemia is indicated, the operational flow may proceed back to block 202 whereby the stimulation device 100 monitors the glucose levels of the patient on a continual (e.g., periodic) basis. In the event hypoglycemia or hyperglycemia is indicated, the operational flow proceeds to block 212.

At block 212, the glycemia monitor 310 (or some other suitable functional component of the device 100 such as a neurostimulation controller) may select parameters for neurostimulation based on one or more characteristics of the indicated glycemic condition. In some aspects, depending on whether the characterization of the glycemic condition indicates hypoglycemia or hyperglycemia, a decision may be made to provide stimulation of parasympathetic innervation, sympathetic innervation, or a combination of the two. Here, in an embodiment where the stimulation device 100 is coupled to different stimulation leads (e.g., neurological lead 104) a decision may be made regarding which leads are to be used for the stimulation operation. For example, if hypoglycemia is indicated, the parasympathetic system may be activated (e.g., to balance out an autonomic sympathetic response) and/or the sympathetic system deactivated. In this case, the heart rate of the patient may decrease as a result of the stimulation of one or more parasympathetic nerves. Conversely, if hyperglycemia is indicated, the sympathetic system may be activated (e.g., to balance out an autonomic parasympathetic response) and/or the parasympathetic system may be deactivated. In this case, the heart rate of the patient may increase as a result of the stimulation of one or more sympathetic nerves.

In addition, signal parameters such as signal frequency and/or signal amplitude may be selected based on the indicated glycemic condition. For example, different parameters may be used for parasympathetic innervation versus sympathetic innervation.

In some implementations the glycemia monitor 310 may select one or more of these parameters based on the severity of the glycemic condition. For example, if the glycemic condition is not severe (e.g., as indicated by detection of a relatively small change in the value of a given cardiac feature at block 208), a relatively small signal magnitude and/or a relatively low frequency may be selected for the stimulation operation. Conversely, if the glycemic condition is relatively severe (e.g., as indicated by detection of a relatively large change in the value of a given cardiac feature at block 208), a relatively large signal magnitude and/or a relatively high frequency may be selected for the stimulation operation.

Also, in some aspects the length of time that a stimulation signal is to be applied may be based on the glycemic condition. For example, the glycemia monitor 310 may designate stimulation duration times based on whether hypoglycemia or hyperglycemia is indicated and/or based on the severity of the glycemic condition.

As represented by block 214, the stimulation device 100 may then generate one or more stimulation signals to stimulate the patient's nervous system. To this end, the stimulation device 100 may include a neurostimulation signal generator 312 (e.g., comprising a pulse generator) that is coupled to at least one nerve stimulation lead (e.g., neurological lead 104). Here, the at least one nerve stimulation lead may be implanted to stimulate one or more parasympathetic nerves and/or implanted to stimulate one or more sympathetic nerves. In some implementations, the signal generator 312 may be configured to generate a bi-polar square wave or some other waveform suitable for nerve stimulation.

During and/or after stimulation of the nervous system, the stimulation device 100 may monitor cardiac activity (e.g., by processing acquired IEGM data) to determine the effect of the stimulation on the patient's condition and adapt the stimulation accordingly. For example, the stimulation may be modified if the severity of the glycemic condition has changed, if the patient's heart rate has changed, if there has been a change in the quantity or severity of observed PVCs, and so on. As a specific example, if the stimulation has mitigated the severity of the response to the glycemic condition, the amplitude and/or the frequency of the stimulation signal may be decreased. Similarly, if hypoglycemia or hyperglycemia is no longer indicated, the application of stimulation may be terminated. In any event, the stimulation device 100 may continue to monitor the patient's condition over time and apply an appropriate level of neurostimulation whenever it is warranted.

As mentioned above, in some implementations the teaching herein may be implemented in an implantable cardiac device that is used to monitor and/or or treat cardiac various conditions. The following description sets forth an exemplary implantable cardiac device (e.g., a stimulation device such as an implantable cardioverter defibrillator, a pacemaker, etc.) that is capable of being used in connection with the various embodiments that are described herein. It is to be appreciated and understood that other devices, including those that are not necessarily implantable, can be used and that the description below is given, in its specific context, to assist the reader in understanding, with more clarity, the embodiments described herein.

FIG. 5 shows an exemplary implantable cardiac device 500 in electrical communication with a patient's heart H by way of three leads 504, 506, and 508, suitable for delivering multi-chamber stimulation and shock therapy. To sense atrial cardiac signals and to provide right atrial chamber stimulation therapy, the device 500 is coupled to an implantable right atrial lead 504 having, for example, an atrial tip electrode 520, which typically is implanted in the patient's right atrial appendage or septum. FIG. 5 also shows the right atrial lead 504 as having an optional atrial ring electrode 521.

To sense left atrial and ventricular cardiac signals and to provide left chamber pacing therapy, the device 500 is coupled to a coronary sinus lead 506 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 506 is designed to receive atrial and ventricular cardiac signals and to deliver left ventricular pacing therapy using, for example, a left ventricular tip electrode 522 and, optionally, a left ventricular ring electrode 523; provide left atrial pacing therapy using, for example, a left atrial ring electrode 524; and provide shocking therapy using, for example, a left atrial coil electrode 526 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 500 is also shown in electrical communication with the patient's heart H by way of an implantable right ventricular lead 508 having, in this implementation, a right ventricular tip electrode 528, a right ventricular ring electrode 530, a right ventricular (RV) coil electrode 532 (or other electrode capable of delivering a shock), and a superior vena cava (SVC) coil electrode 534 (or other electrode capable of delivering a shock). Typically, the right ventricular lead 508 is transvenously inserted into the heart H to place the right ventricular tip electrode 528 in the right ventricular apex so that the RV coil electrode 532 will be positioned in the right ventricle and the SVC coil electrode 534 will be positioned in the superior vena cava. Accordingly, the right ventricular lead 508 is capable of sensing or receiving cardiac signals, and delivering stimulation in the form of pacing and shock therapy to the right ventricle.

The device 500 is also shown in electrical communication with one or more neurostimulation leads 510 each of which includes, for example, one or more stimulation electrodes 544. As discussed herein, the electrode(s) 544 may be positioned on or near one or more nerves of a patient.

It should be appreciated that the device 500 may connect to leads other than those specifically shown. In addition, the leads connected to the device 500 may include components other than those specifically shown. For example, a lead may include other types of electrodes, sensors or devices that serve to otherwise interact with a patient or the surroundings.

FIG. 6 depicts an exemplary, simplified block diagram illustrating sample components of the device 500. The device 500 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

Housing 600 for the device 500 is often referred to as the “can”, “case” or “case electrode”, and may be programmably selected to act as the return electrode for all “unipolar” modes. The housing 600 may further be used as a return electrode alone or in combination with one or more of the coil electrodes 526, 532 and 534 for shocking purposes. Housing 600 further includes a connector (not shown) having a plurality of terminals 601, 602, 604, 605, 606, 608, 612, 614, 616 and 618 (shown schematically and, for convenience, the names of the electrodes to which they are connected are shown next to the terminals).

The connector may be configured to include various other terminals depending on the requirements of a given application. For example, one or more terminals 621 may be coupled to one or more implantable neurostimulation leads (e.g., lead 510) as discussed herein.

To achieve right atrial sensing and pacing, the connector includes, for example, a right atrial tip terminal (AR TIP) 602 adapted for connection to the right atrial tip electrode 520. A right atrial ring terminal (AR RING) 601 may also be included and adapted for connection to the right atrial ring electrode 521. To achieve left chamber sensing, pacing, and shocking, the connector includes, for example, a left ventricular tip terminal (VL TIP) 604, a left ventricular ring terminal (VL RING) 605, a left atrial ring terminal (AL RING) 606, and a left atrial shocking terminal (AL COIL) 608, which are adapted for connection to the left ventricular tip electrode 522, the left ventricular ring electrode 523, the left atrial ring electrode 524, and the left atrial coil electrode 526, respectively.

To support right chamber sensing, pacing, and shocking, the connector further includes a right ventricular tip terminal (VR TIP) 612, a right ventricular ring terminal (VR RING) 614, a right ventricular shocking terminal (RV COIL) 616, and a superior vena cava shocking terminal (SVC COIL) 618, which are adapted for connection to the right ventricular tip electrode 528, the right ventricular ring electrode 530, the RV coil electrode 532, and the SVC coil electrode 534, respectively.

At the core of the device 500 is a programmable microcontroller 620 that controls the various modes of stimulation therapy. As is well known in the art, microcontroller 620 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 620 includes the ability to process or monitor input signals (data or information) as controlled by a program code stored in a designated block of memory. The type of microcontroller is not critical to the described implementations. Rather, any suitable microcontroller 620 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander et al.) and 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 6 also shows pulse generators 622 (e.g., an atrial pulse generator and a ventricular pulse generator) that generate pacing stimulation pulses for delivery by the right atrial lead 504, the coronary sinus lead 506, the right ventricular lead 508, or some combination of these leads via an electrode configuration switch 626. It is understood that in order to provide stimulation therapy in each of the four chambers of the heart, the atrial and ventricular pulse generators 622 may include dedicated, independent pulse generators, multiplexed pulse generators, or shared pulse generators. The pulse generators 622 are controlled by the microcontroller 620 via appropriate control signals 628 to trigger or inhibit the stimulation pulses.

Microcontroller 620 further includes timing control circuitry 632 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 620 further includes an arrhythmia detector 634. The arrhythmia detector 634 may be utilized by the device 500 for determining desirable times to administer various therapies. The arrhythmia detector 634 may be implemented, for example, in hardware as part of the microcontroller 620, or as software/firmware instructions programmed into the device 500 and executed on the microcontroller 620 during certain modes of operation.

Microcontroller 620 may include a morphology discrimination module 636, a capture detection module (not shown) and an auto sensing module (not shown). These modules are optionally used to implement various exemplary recognition algorithms or methods. The aforementioned components may be implemented, for example, in hardware as part of the microcontroller 620, or as software/firmware instructions programmed into the device 500 and executed on the microcontroller 620 during certain modes of operation.

The electrode configuration switch 626 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 626, in response to a control signal 642 from the microcontroller 620, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 644 and ventricular sensing circuits (VTR. SENSE) 646 may also be selectively coupled to the right atrial lead 504, coronary sinus lead 506, and the right ventricular lead 508, through the switch 626 for detecting the presence of cardiac activity in each of the four chambers of the heart. Accordingly, the atrial and ventricular sensing circuits 644 and 646 may include dedicated sense amplifiers, multiplexed amplifiers, or shared amplifiers. Switch 626 determines the “sensing polarity” of the cardiac signal by selectively closing the appropriate switches, as is also known in the art. In this way, the clinician may program the sensing polarity independent of the stimulation polarity. The sensing circuits (e.g., circuits 644 and 646) are optionally capable of obtaining information indicative of tissue capture.

Each sensing circuit 644 and 646 preferably employs one or more low power, precision amplifiers with programmable gain, automatic gain control, bandpass filtering, a threshold detection circuit, or some combination of these components, to selectively sense the cardiac signal of interest. The automatic gain control enables the device 500 to deal effectively with the difficult problem of sensing the low amplitude signal characteristics of atrial or ventricular fibrillation.

The outputs of the atrial and ventricular sensing circuits 644 and 646 are connected to the microcontroller 620, which, in turn, is able to trigger or inhibit the atrial and ventricular pulse generators 622 in a demand fashion in response to the absence or presence of cardiac activity in the appropriate chambers of the heart. Furthermore, as described herein, the microcontroller 620 is also capable of analyzing information output from the sensing circuits 644 and 646, a data acquisition system 652, or both. This information may be used to determine or detect whether and to what degree tissue capture has occurred and to program a pulse, or pulses, in response to such determinations. The sensing circuits 644 and 646, in turn, receive control signals over signal lines 648 and 650, respectively, from the microcontroller 620 for purposes of controlling the gain, threshold, polarization charge removal circuitry (not shown), and the timing of any blocking circuitry (not shown) coupled to the inputs of the sensing circuits 644 and 646 as is known in the art.

For arrhythmia detection, the device 500 utilizes the atrial and ventricular sensing circuits 644 and 646 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 634 of the microcontroller 620 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 652. The data acquisition system 652 is configured (e.g., via signal line 656) to acquire intracardiac electrogram (“IEGM”) signals or other signals, convert the raw analog data into a digital signal, and store the digital signals for later processing, for telemetric transmission to an external device 654, or both. For example, the data acquisition system 652 may be coupled to the right atrial lead 504, the coronary sinus lead 506, the right ventricular lead 508 and other leads through the switch 626 to sample cardiac signals across any pair of desired electrodes.

The data acquisition system 652 also may be coupled to receive signals from other input devices. For example, the data acquisition system 652 may sample signals received via the terminal 621 or signals from a physiologic sensor 670 or other components shown in FIG. 6 (connections not shown).

The microcontroller 620 is further coupled to a memory 660 by a suitable data/address bus 662, wherein the programmable operating parameters used by the microcontroller 620 are stored and modified, as required, in order to customize the operation of the device 500 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 652), which data may then be used for subsequent analysis to guide the programming of the device 500.

Advantageously, the operating parameters of the implantable device 500 may be non-invasively programmed into the memory 660 through a telemetry circuit 664 in telemetric communication via communication link 665 with the external device 654, such as a programmer, transtelephonic transceiver, a diagnostic system analyzer or some other device. The microcontroller 620 activates the telemetry circuit 664 with a control signal (e.g., via bus 668). The telemetry circuit 664 advantageously allows intracardiac electrograms and status information relating to the operation of the device 500 (as contained in the microcontroller 620 or memory 660) to be sent to the external device 654 through an established communication link 665.

The device 500 can further include one or more physiologic sensors 670. In some embodiments the device 500 may include a “rate-responsive” sensor that may provide, for example, information to aid in adjustment of pacing stimulation rate according to the exercise state of the patient. One or more physiologic sensors 670 (e.g., a pressure sensor) may further be used to detect changes in cardiac output, changes in the physiological condition of the heart, or diurnal changes in activity (e.g., detecting sleep and wake states). Accordingly, the microcontroller 620 responds by adjusting the various pacing parameters (such as rate, A-V Delay, V-V Delay, etc.) at which the atrial and ventricular pulse generators 622 generate stimulation pulses.

While shown as being included within the device 500, it is to be understood that a physiologic sensor 670 may also be external to the device 500, yet still be implanted within or carried by the patient. Examples of physiologic sensors that may be implemented in conjunction with the device 500 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 670 may optionally include one or more of components to help detect movement (via, e.g., a position sensor or an accelerometer) and minute ventilation (via an MV sensor) in the patient. Signals generated by the position sensor and MV sensor may be passed to the microcontroller 620 for analysis in determining whether to adjust the pacing rate, etc. The microcontroller 620 may thus monitor the signals for indications of the patient's position and activity status, such as whether the patient is climbing up stairs or descending down stairs or whether the patient is sitting up after lying down.

The device 500 additionally includes a battery 676 that provides operating power to all of the circuits shown in FIG. 6. For a device 500 which employs shocking therapy, the battery 676 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 676 also desirably has a predictable discharge characteristic so that elective replacement time can be detected. Accordingly, the device 500 preferably employs lithium or other suitable battery technology.

The device 500 can further include magnet detection circuitry (not shown), coupled to the microcontroller 620, to detect when a magnet is placed over the device 500. A magnet may be used by a clinician to perform various test functions of the device 500 and to signal the microcontroller 620 that the external device 654 is in place to receive data from or transmit data to the microcontroller 620 through the telemetry circuit 664.

The device 500 further includes an impedance measuring circuit 678 that is enabled by the microcontroller 620 via a control signal 680. The known uses for an impedance measuring circuit 678 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 500 has been implanted; measuring stroke volume; and detecting the opening of heart valves, etc. The impedance measuring circuit 678 is advantageously coupled to the switch 626 so that any desired electrode may be used.

In the case where the device 500 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 620 further controls a shocking circuit 682 by way of a control signal 684. The shocking circuit 682 generates shocking pulses of low (e.g., up to 0.5 J), moderate (e.g., 0.5 J to 10 J), or high energy (e.g., 11 J to 40 J), as controlled by the microcontroller 620. Such shocking pulses are applied to the patient's heart H through, for example, two shocking electrodes and as shown in this embodiment, selected from the left atrial coil electrode 526, the RV coil electrode 532 and the SVC coil electrode 534. As noted above, the housing 600 may act as an active electrode in combination with the RV coil electrode 532, as part of a split electrical vector using the SVC coil electrode 534 or the left atrial coil electrode 526 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 620 is capable of controlling the synchronous or asynchronous delivery of the shocking pulses.

As mentioned above, the device 500 may include several components that provide functionality relating to neurostimulation as taught herein. For example, one or more of the switch 626, the sense circuits 644, 646, and the data acquisition system 652 may acquire cardiac signals that are used by an IEGM processing component 637 to provide IEGM data as discussed above. This IEGM data may be stored in the data memory 660. In addition, a neuro-signal generator 640 may generate neurostimulation signals as taught herein. Here, the microcontroller 620 may provide one or more control signals 630 to the neuro-signal generator 640 to control the timing (e.g., start and stop times) and other parameters (e.g., amplitude, waveshape, and frequency) of the neurostimulation signals.

The microcontroller 620 (e.g., a processor providing signal processing functionality) also may implement or support at least a portion of the neurostimulation-related functionality discussed herein. For example, a glycemia monitor 638 may perform glycemia-related operations as described above (e.g., determining whether a hypoglycemia condition or a hyperglycemia condition exists). In addition, a neurological stimulation controller 639 may perform neurostimulation operations such as, for example, determining which form of innervation to use based on the current glycemic condition and the parameters for the neurostimulation signals.

It should be appreciated that various modifications may be incorporated into the disclosed embodiments based on the teachings herein. For example, the structure and functionality taught herein may be incorporated into types of devices other than the specific types of devices described above. Also, different types of stimulation signals may be applied to the nervous system consistent with the teachings herein. In addition, neurostimulation signals may be applied to other locations consistent with the teachings herein. Furthermore, a determination to apply neurostimulation may be made based on glycemic conditions that are indicated in other ways consistent with the teachings herein.

It should be appreciated from the above that the various structures and functions described herein may be incorporated into a variety of apparatuses (e.g., a stimulation device, a lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure as defined by any claims associated herewith.

What is claimed is:

1. A method of neurostimulation, comprising:
sensing cardiac activity;
generating intracardiac electrogram data based on the sensed cardiac activity;
identifying a glycemic condition based on the intracardiac electrogram data; and
stimulating at least one nerve based on the identified glycemic condition.

2. The method of claim 1, wherein the glycemic condition comprises hypoglycemia or hyperglycemia.

3. The method of claim 1, further comprising:
characterizing the glycemic condition; and
determining whether to employ parasympathetic innervation or sympathetic innervation for the stimulation based on the characterization of the glycemic condition.

4. The method of claim 1, wherein the stimulation comprises providing parasympathetic innervation to counteract an autonomic sympathetic response associated with the glycemic condition.

5. The method of claim 1, wherein the stimulation comprises providing sympathetic innervation to counteract an autonomic parasympathetic response associated with the glycemic condition.

6. The method of claim 1, wherein the stimulation comprises enhancing or inhibiting an autonomic nervous system response.

7. The method of claim 1, wherein:
the glycemic condition comprises hypoglycemia; and
the stimulation comprises activating a parasympathetic response and/or deactivating a sympathetic response.

8. The method of claim 1, wherein:
the glycemic condition comprises hyperglycemia; and
the stimulation comprises activating a sympathetic response and/or deactivating a parasympathetic response.

9. The method of claim 1, wherein the stimulation causes a change in heart rate.

10. The method of claim 1, further comprising:
determining a severity of the glycemic condition; and
specifying at least one signal parameter for the stimulation based on the severity of the glycemic condition.

11. The method of claim 10, wherein the at least one signal parameter comprises frequency and/or amplitude.

12. The method of claim 1, further comprising:
determining whether the stimulation has mitigated the glycemic condition; and
adapting the stimulation based on the determination.

13. The method of claim 1, wherein the identification of a glycemic condition comprises detecting a variance in at least one of the group consisting of: cardiac event duration, cardiac event amplitude, and cardiac event dispersion.

14. The method of claim 1, wherein the identification of a glycemic condition comprises generating an indication relating to variance of a cardiac feature represented by the intracardiac electrogram data.

15. The method of claim 14, wherein the cardiac feature relates to at least one of the group consisting of: QT interval time period, T-wave timing, R-wave amplitude, R-wave area, and T-wave area.

16. The method of claim 14, further comprising:
determining whether the cardiac feature is associated with an intrinsic cardiac event or a paced cardiac event; and
normalizing the variance indication based on the determination.

17. The method of claim 1, wherein the at least one nerve carries neurotransmitters associated with at least one cardiac autonomic response.

18. The method of claim 1, wherein the stimulation of the at least one nerve comprises applying a stimulation signal to a lead implanted adjacent to at least one or the group consisting of: a vagus nerve, an epicardial fat pad, a cardiac nerve, and a spinal cord vertebra.

19. A stimulation apparatus, comprising:
a cardiac sensing circuit that senses cardiac signals;
an electrogram processor that generates intracardiac electrogram data based on the sensed cardiac signals;
a glycemia monitor that identifies a glycemic condition based on the intracardiac electrogram data; and
a neurostimulation signal generator that generates at least one signal for stimulating at least one nerve based on the identified glycemic condition.

20. The apparatus of claim 19, wherein the glycemia monitor is further configured to:
determine a severity of the glycemic condition; and
specify frequency and/or amplitude for the at least one signal based on the severity of the glycemic condition.
